(19) United States
(12) Patent Application Publication
(10) Pub. No.: US 2011/0009692 A1
(43) Pub. Date: Jan. 13, 2011

(54) NITRIC OXIDE GENERATION TO TREAT FEMALE SEXUAL DYSFUNCTION

(76) Inventor: Yossi Gross, Moshav Mazor (IL)

Correspondence Address: SUGHRUE MION, PLLC, 2100 PENNSYLVANIA AVENUE, N.W., SUITE 800, WASHINGTON, DC, 20037, US

(21) Appl. No.: 12/810,763

(22) PCT Filed: Dec. 28, 2008
(86) PCT No.: PCT/IL08/01686

§ 371 Date: Sep. 24, 2010

Related U.S. Application Data
(60) Provisional application No. 61/009,306, filed on Dec. 26, 2007.

(51) Int. Cl. A61F 5/00; A61N 1/00

(52) U.S. Cl. 600/38; 607/138

(57) ABSTRACT

Apparatus for treating female sexual dysfunction of a subject is provided. A manual insertion device (20) is configured to be inserted by the subject into a vagina of the subject and removed by the subject from the vagina of the subject. At least one electrode (24) is coupled to the insertion device. A control unit (22) is configured to chronically increase nitric oxide production by tissue of the subject by driving the electrode to drive an electric current into the vagina. Other embodiments are also described.

### CROSS-REFERENCES TO RELATED APPLICATIONS

The present application claims the benefit of U.S. Provisional Patent Application 61/009,306 to Gross, filed Dec. 26, 2007, which is incorporated herein by reference.

### FIELD OF THE INVENTION

The present invention generally relates to medical apparatus. Specifically, the present invention relates to an electrode device for treating female sexual dysfunction.

### BACKGROUND OF THE INVENTION

Female sexual dysfunction is a condition in which a subject experiences discomfort, pain, or decreased pleasure during sexual activity. Hormonal changes in a subject who has undergone menopause may result in the subject experiencing symptoms that are indicative of female sexual dysfunction.

PCT Publication WO 07/059990 to Boyd et al. describes a compressible electrode for the stimulation of the musculature of the pelvic floor complex, e.g., for the treatment of anterior and posterior pelvic floor muscle dysfunction, which is reversibly compressible and has electro-conductive elements. The compressible electrode is described as being used with all the usual control units and treatment regimes for the electro-stimulation of the musculature and nerves of the vagina and/or anus. The compressible electrode may be inserted into the vagina or anus through the use of an applicator. In the compressed state, the compressible electrode may be of tampon proportions and after use may easily be removed.

PCT Publication WO 05/092439 to Fox et al. describes a method for applying light energy, preferably at a wavelength of 884 nanometers, to the genitalia area of male and female humans and animals to treat erectile sexual dysfunction. The light is described as causing the release of nitric oxide into the erectile genitalia tissue, and the nitric oxide as causing the smooth erectile tissue to relax and engorge, thereby facilitating erection. Separately, structured applicators for males and females are described as optimizing the degree of light energy penetration and the amount of nitric oxide released according to the separate physiology of males and females. The method is described as being used to augment a pharmacologically induced release of nitric oxide.

An article by Gragasin F S et al., entitled, “The neurovascular mechanism of clitoral erection: nitric oxide and cGMP-stimulated activation of BKCa channels,” The FASEB Journal 18:1382-1391, 2004, describes an investigation to evaluate two hypotheses: 1) NO and sildenafil cause clitoral relaxation through a PKG- and BKCa channel-dependent mechanism; and 2) electrical field stimulation (EFS), which is described by the authors as being a common experimental means of simulating sexual stimulation in erectile tissue, causes NO synthesis and induces relaxation of the rat clitoris via this same mechanism. The authors state that NO is a potent relaxer of clitoral tissue, reminiscent of its effects on penile tissue and that EFS induces measurable clitoral NO synthesis.

An article by Munarriz K R et al., entitled, “A review of the physiology and pharmacology of peripheral (vaginal and clitoral) female genital arousal in the animal model,” J Urol. 170(2 Pt 2):S40-4, 2003, describes a review of contemporary scientific data concerning the physiology and pharmacology of peripheral female genital arousal responses in the animal (rabbit and rat) model. The authors state that nitric oxide appears to be a key pathway mediating clitoral smooth muscle relaxation.

An article by Pacher et al., entitled, “Topical administration of novel nitric oxide donor, linear polyethylenimine-nitric oxide/nucleophile adduct (DS1), selectively increases vaginal blood flow in anesthetized rats,” International Journal of Impotence Research 15:461-464, 2003, describes a study to test the effects of a topical administration of a nitric oxide donor, linear polyethylenimine-nitric oxide/nucleophile adduct (DS1), on vaginal blood flow and hemodynamics in rats. The authors conclude that topical application of nitric oxide donors such as DS1 is useful for the treatment of female sexual dysfunction that develops due to an impairment of local blood flow supply to the vaginal tissue.

Pfizer (New York City, N.Y.) manufactures Cleocin®, a clindamycin phosphate vaginal cream described as an intravaginal treatment for vaginal odor caused by bacterial vaginosis.

Galderma (France, Canada, Brazil) manufactures metronidazole marketed under the trade name Metrogel®, which is described as a vaginal gel used for the treatment of bacterial vaginosis.

The following patents and patent applications, may be of interest:

U.S. Pat. No. 7,229,403 to Schock et al.

U.S. Pat. No. 7,206,637 to Salo

U.S. Pat. No. 6,939,345 to Ken Knight et al.

U.S. Pat. No. 6,896,651 to Gross et al.

U.S. Pat. No. 6,871,092 to Piccone

U.S. Pat. No. 6,865,416 to Dev et al.

U.S. Pat. No. 6,862,480 to Cohen et al.

U.S. Pat. No. 6,845,267 to Harrison et al.

U.S. Pat. No. 6,824,561 to Soykan et al.

U.S. Pat. No. 6,810,286 to Donovan et al.

U.S. Pat. No. 6,741,895 to Gafni et al.

U.S. Pat. No. 6,485,524 to Strecker

U.S. Pat. No. 6,479,045 to Bologna et al.

U.S. Pat. No. 6,463,323 to Conrad-Vlasak et al.

U.S. Pat. No. 6,432,037 to Eini et al.

U.S. Pat. No. 6,347,247 to Dev et al.

U.S. Pat. No. 6,245,103 to Stinson

U.S. Pat. No. 6,200,259 to March

U.S. Pat. No. 6,139,538 to Houghton et al.

U.S. Pat. No. 6,086,527 to Talpade

U.S. Pat. No. 6,058,331 to King

U.S. Pat. No. 6,030,375 to Anderson et al.

U.S. Pat. No. 5,906,641 to Thompson et al.

U.S. Pat. No. 5,800,502 to Boutos

U.S. Pat. No. 5,800,501 to Sherlock et al.

U.S. Pat. No. 5,324,323 to Bui

U.S. Pat. No. 5,046,511 to Mauer et al.

U.S. Pat. No. 5,830,848 to Harrison et al.

U.S. Pat. No. 4,827,946 to Kaali et al.

U.S. Design Pat. No. 415835 to Malewicz et al.

U.S. Patent Application Publication 1998/34677 to Hilburg et al.

U.S. Patent Application Publication 2002/0103454 to Sackner et al.

U.S. Patent Application Publication 2003/0036773 to Whitehurst et al.

U.S. Patent Application Publication 2003/0204206 to Padua et al.

U.S. Patent Application Publication 2003/057264 to Geiser et al.

U.S. Patent Application Publication 2004/0039417 to Soykan et al.

U.S. Patent Application Publication 2006/0276844 to Alon et al.

U.S. Patent Application Publication 2007/0196428 to Glauser et al.

U.S. Patent Application Publication 2007/0248676 to Stamler et al.

PCT Publication WO 00/002501 to Benjamin et al.

PCT Publication WO 04/014456 to Allen et al.

PCT Publication WO 06/094273 to White et al.

PCT Publication WO 07/106533 to Stem et al.

PCT Publication WO 07/113833 to Cahan et al.

PCT Publication WO 2006/064503 to Belsky et al.

PCT Publication WO 2006/123346 to Alon et al.

European Patent Application Publication EP 0 109 935 A1 to Chaimillot et al.

The following articles may be of interest:

Gorodeski G I, “NO increases permeability of cultured human cervical epithelia by cGMP-mediated increase in G-actin,” Am J Physiol Cell Physiol 278:942-952 2000

Morlin et al., “Nitric oxide increases endocervical secretion at the ovulatory phase in the female,” Acta Obstet Gynecol Scand 84:883-886 2005

Morlin et al., “Nitric oxide induces endometrial secretion at implantation time,” Acta Obstet Gynecol Scand 84:1029-1034 2005

Norman et al., “Nitric oxide in the human uterus,” Reviews of Reproduction 1:61-68 1996

Sobel et al., “Efficacy of clindamycin vaginal ovule (3-day treatment) vs. clindamycin vaginal cream (7-day treatment) in bacterial vaginosis,” Infect Dis Obstet Gynecol 9:9-15, 2001

Vachon P S et al., “Increases in clitoral and vaginal blood flow following clitoral and pelvic plexus nerve stimulations in the female rat,” Int J Impot Res. 12(1):53-7, 2000

### SUMMARY OF THE INVENTION

In some embodiments of the present invention, a subject is identified as suffering from female sexual dysfunction. An insertion device is manually inserted into the subject's vagina to chronically increase nitric oxide production by tissue of the subject. The device is typically inserted by the subject in accordance with a schedule unrelated to timing of sexual intercourse. The production of nitric oxide typically causes healing and restoration of vaginal tissue that is damaged with menopause, and that could otherwise be treated with hormone replacement therapy.

Typically, the manual insertion device is tampon-shaped and comprises a control unit and one or more electrodes. The control unit is configured to drive the electrodes to drive an electrical current into the subject's vagina in order to chronically increase nitric oxide production by tissue of the subject.

In some embodiments, the device comprises a panty liner (not inserted into the subject), which comprises a control unit and one or more electrodes. The control unit is configured to drive the electrodes to drive an electrical current into the subject's vagina in order to chronically increase nitric oxide production by tissue of the subject.

In some embodiments, one or more electrode units are surgically implanted into tissue of a pelvic site in or adjacent to the vagina. In such embodiments, the control unit of the manual insertion device or of the panty liner is configured to wirelessly drive the electrode units to drive a current into the pelvic site.

In some embodiments of the invention, the manual insertion device is configured to administer a drug. For such embodiments, the manual insertion device comprises a reservoir from which the drug is administered. Alternatively or additionally, the control unit is configured to facilitate the administration of the drug by controlling one or more of the electrodes (or other electrodes) to iontophoretically drive the drug into the tissue of the subject.

In some embodiments, the outer surface of the manual insertion device is coated with a drug, and insertion of the device into the subject's vagina releases a portion of the drug. For some applications, the drug within the reservoir includes a hormone. Alternatively or additionally, the manual insertion device is configured to control the pH, and/or an odor of the vagina. In some applications, the substance for controlling pH and/or odor is administered to the subject via the reservoir. In some embodiments, water is delivered to the vagina via the reservoir. The electrolysis of the water causes the release of oxygen inside the vagina, and the presence of oxygen acts to control odor, and/or control pH of the vagina. In some embodiments, a pharmaceutical substance or an odor-controlling and/or a pH-controlling substance is delivered to the vagina, via the reservoir, in addition to water. The electrodes are configured to electrolyze the water and release oxygen from the water. The electrolysis of the water further enhances the dispersal of the substance (i.e., the release of the substance into the vagina). Alternatively or additionally, the outer surface of the manual insertion device is coated with the substance for controlling pH and/or odor.

In some embodiments, the manual insertion device generates a current for treatment of urge incontinence, in addition to the current described hereinabove for treatment of female sexual dysfunction. For example, the manual insertion device may stimulate a muscle such as a pelvic floor muscle to treat urge incontinence. Alternatively or additionally, the manual insertion device stimulates a nerve to treat the urge incontinence, optionally as a consequence of applying the current to the muscle.

There is therefore provided, in accordance with an embodiment of the invention, apparatus for treating female sexual dysfunction of a subject, including:
a manual insertion device configured to be inserted by the subject into a vagina of the subject and removed by the subject from the vagina of the subject;
at least one electrode coupled to the insertion device; and
a control unit, configured to chronically increase nitric oxide production by tissue of the subject by driving the electrode to drive an electric current into the vagina.

In an embodiment, the manual insertion device is configured to be inserted into the vagina of the subject in accordance with a schedule unrelated to timing of sexual intercourse of the subject.

In an embodiment, the manual insertion device is configured to be inserted into the vagina of the subject for between half an hour and two hours in a day, in accordance with a schedule unrelated to timing of sexual intercourse of the subject.

In an embodiment, the manual insertion device is configured to be inserted into the vagina of the subject for between two hours and five hours in a day, in accordance with a schedule unrelated to timing of sexual intercourse of the subject.

In an embodiment, the total length of the apparatus is 4-10 cm.

In an embodiment, the insertion device is configured to administer a drug.

In an embodiment, the drug includes a hormone selected from the group consisting of: estrogen and progesterone, and wherein the insertion device is configured to administer the selected hormone.

In an embodiment, the insertion device is configured to control a pH of the vagina.

In an embodiment, the apparatus includes water, wherein the insertion device is configured to administer the water to the subject, and wherein the insertion device is configured to control the pH of the vagina by releasing oxygen into the vagina by electrolyzing the water.

In an embodiment, the apparatus includes a pH-controlling substance, wherein the insertion device is configured to administer the pH-controlling substance to the vagina.

In an embodiment, the insertion device is configured to iontophoretically deliver the substance into tissue of the vagina.

In an embodiment, the apparatus includes water, wherein the insertion device is configured to administer the water to the vagina of the subject, and wherein the insertion device is configured to control the pH of the vagina by releasing the substance into the vagina by electrolyzing the water.

In an embodiment, the insertion device is configured to control an odor of the vagina.

In an embodiment, the apparatus includes water, wherein the insertion device is configured to administer the water to the subject, and wherein the insertion device is configured to control the odor of the vagina by releasing oxygen the vagina by electrolyzing the water.

In an embodiment, the apparatus includes an odor-controlling substance, wherein the insertion device is configured to administer the odor-controlling substance to the subject.

In an embodiment, the insertion device is configured to iontophoretically deliver the substance into tissue of the subject.

In an embodiment, the apparatus includes water, wherein the insertion device is configured to administer the water to the subject, and wherein the insertion device is configured to control the odor of the vagina by releasing the substance into the vagina by electrolyzing the water.

In an embodiment, the control unit is configured to configure the electric current to have a frequency that is between 8 Hz and 20 Hz.

In an embodiment, the control unit is configured to configure the electric current to have a frequency that is between 10 Hz and 15 Hz.

In an embodiment, the control unit is configured to configure the electric current to have an amplitude that is between 1 mA and 5 mA.

In an embodiment, the control unit is configured to configure the electric current to have an amplitude that is between 2 and 3 mA.

In an embodiment, the control unit is configured to drive an electric current to treat urge incontinence.

In an embodiment, the control unit is configured to chronically increase the nitric oxide production by using a first signal protocol, and to treat the urge incontinence using a second signal protocol, different from the first signal protocol.

In an embodiment, the control unit is configured to drive the electric current to treat the urge incontinence into a pelvic floor muscle of the subject.

In an embodiment, the manual insertion device includes a compressible material.

In an embodiment, the manual insertion device is configured:

to be inserted into the vagina by being compressed, and

upon having been inserted into the vagina, to conform to a shape of a vaginal cavity of the subject, by expanding.

In an embodiment, the apparatus further includes a conductive gel, and the electrode is coupled to the conductive gel.

In an embodiment, the conductive gel is configured to reduce a level of discomfort associated with the driving of the current into the vagina.

In an embodiment, the conductive gel is configured to facilitate delivery of the current into vaginal tissue that is deeper than a depth of the vaginal tissue to which the current would be delivered not in the presence of the gel.

There is additionally provided, in accordance with an embodiment of the invention, apparatus for treating female sexual dysfunction of a subject, including:

at least one electrode, configured to be surgically implanted at a pelvic site of the subject; and

a control unit, configured to chronically increase nitric oxide production by tissue of the subject by driving the electrode to drive an electric current into the pelvic site.

In an embodiment, the control unit comprises a surgically implantable control unit.

In an embodiment, the apparatus includes a manual insertion device that includes the control unit, the manual insertion device being configured to be inserted by the subject into the vagina and removed by the subject from the vagina.

In an embodiment, the control unit is configured to wirelessly drive the electrode to drive the current.

In an embodiment, the apparatus includes a panty liner that includes the control unit.

In an embodiment, the control unit is configured to wirelessly drive the electrode to drive the current.

There is still further provided, in accordance with an embodiment of the invention apparatus for treating female sexual dysfunction of a subject, including:

a panty liner configured to be inserted by the subject into underwear of the subject;

at least one electrode coupled to the panty liner; and

a control unit, configured to chronically increase nitric oxide production by tissue of the subject by driving the electrode to drive an electric current into the vagina.

There is further provided, in accordance with an embodiment of the invention, apparatus for treating menorrhagia of a subject, including:

a manual insertion device configured to be inserted by the subject into a vagina of the subject and removed by the subject from the vagina of the subject;

at least one electrode coupled to the insertion device; and

a control unit, configured to decrease nitric oxide production by tissue of the subject by driving the electrode to drive an electric current into the vagina.

In an embodiment, the control unit is configured to drive the current at a frequency greater than 50 Hz.

In an embodiment, the control unit is configured to drive the current into a cervix of the subject.

There is yet further provided, in accordance with an embodiment of the invention, a method for treating female sexual dysfunction of a subject, including:

driving an electric current from a panty liner; and

configuring the electric current to chronically increase nitric oxide production by tissue of the subject.

There is additionally provided, in accordance with an embodiment of the invention, a method for treating female sexual dysfunction of a subject, including:

driving an electric current from within a vagina of the subject; and

configuring the electric current to chronically increase nitric oxide production by tissue of the subject.

There is still further provided, in accordance with an embodiment of the invention, a method, including:

identifying a subject as suffering from female sexual dysfunction; and

treating the sexual dysfunction by driving an electric current from within a vagina of the subject, not within 30 minutes prior to the subject undergoing sexual intercourse.

In an embodiment, driving the electric current includes driving the electric current not within one hour prior to the subject undergoing sexual intercourse.

In an embodiment, driving the electric current includes driving the electric current not within three hours prior to the subject undergoing sexual intercourse.

There is further provided, in accordance with an embodiment of the invention, a method, including:

identifying a subject as suffering from female sexual dysfunction; and

treating the sexual dysfunction by driving an electric current from within a vagina of the subject in accordance with a schedule unrelated to timing of sexual intercourse of the subject.

The present invention will be more fully understood from the following detailed description of embodiments thereof, taken together with the drawings, in which:

### BRIEF DESCRIPTION OF THE DRAWINGS

FIG. 1 is a schematic illustration of an insertion device, in accordance with an embodiment of the present invention;

FIG. 2 is a schematic illustration of an insertion device, in accordance with another embodiment of the present invention;

FIG. 3 is a schematic illustration of an insertion device, with electrodes surgically implanted into tissue of a subject, in accordance with another embodiment of the present invention; and

FIGS. 4A-B are schematic illustrations of panty liners, in accordance with respective embodiments of the present invention.

### DETAILED DESCRIPTION OF EMBODIMENTS

Reference is now made to FIG. 1, which is a schematic illustration of a manual insertion device 20 for insertion into a subject's vagina, in accordance with an embodiment of the present invention. A control unit 22 is coupled to manual insertion device 20, e.g., by being disposed within a proximal body portion 25 or a distal body portion 21 of the device. The control unit is coupled to one or more electrodes 24 via electrical leads 26 and is configured to drive the electrodes to drive an electrical current into the subject's vagina. The device is configured to chronically increase nitric oxide production by tissue of the subject, by driving the current into the vagina.

Typically, manual insertion device 20 is tampon-shaped (the total length of the device being 4-10 cm, for example). In some embodiments, the manual insertion device is made of a compressible material, for example, a sponge-like material. The device is compressed for insertion into the vagina, and upon entry into the vagina the device conforms to the shape of the vaginal cavity by expanding.

Further typically, the subject is identified as suffering from female sexual dysfunction and the device is inserted as a chronic treatment for the female sexual dysfunction. Thus, the device is typically inserted by the subject in accordance with a schedule unrelated to timing of sexual intercourse of the subject. Typically, the device is inserted by the subject for between half an hour and two hours, or between two hours and five hours each day, typically unrelated to a time of the subject undergoing sexual intercourse. For example, the device may be inserted every morning or every evening, and not necessarily within any particular temporal proximity to sexual intercourse, (e.g., not within 30 minutes, one hour or three hours) prior to sexual intercourse. Thus, although the signal protocol used by control unit 22 to drive electrodes 24 may have short-term effects on sexual dysfunction (e.g., such as techniques that are known in the prior art), the manual insertion device is intended for use on a regular basis, unrelated to times of sexual activity, in order to induce long-term physiological changes in the subject.

Typically, control unit 22 drives electrodes 24 to drive an electric current having a frequency of between 8 Hz and 20 Hz, for example, between 10 Hz and 15 Hz. Further typically, the control unit drives each of the electrodes to drive a current having an amplitude of between 1 mA and 5 mA, e.g., between 2 mA and 3 mA. In some embodiments, the control unit drives a current having parameters such as the parameters of currents described in PCT Patent Application No. PCT/IL06/00856 to Gross et al., filed Jul. 25, 2006, which published as WO 07/013065 and is incorporated herein by reference.

In some embodiments, the apparatus comprises a monopolar electrode 24 or single pair of electrodes 24, and control unit 22 drives the electric current into the vagina via the monopolar electrode or single pair of electrode. Alternatively, control unit 22 drives the electric current via a plurality of electrodes 24. In some embodiments, the control unit drives the current via each of the plurality of electrodes in a sequence. For some applications, electrode 24 is coated with conductive gel. In some embodiments, coating the electrode with conductive gel reduces discomfort or pain associated with the delivery of the current into the vagina. Alternatively or additionally, the conductive gel facilitates delivery of the current into vaginal tissue that is deeper than the depth of the tissue to which the current would be delivered not in the presence of the gel.

As appropriate for any given application, electrical leads 26 may connect electrodes 24 to one another, and/or to control unit 22 in a number of different configurations. For some applications, electrodes 24 are disposed annularly and coaxially with respect to distal body portion 21 of manual insertion device 20, as shown in FIG. 1. Alternatively, some or all of electrodes 24 are disposed on proximal body portion 25 of manual insertion device 20.

In some embodiments, manual insertion device 20 is configured to administer a drug. For some applications, the manual insertion device is shaped to define a reservoir 29, from which the drug is administered. In an embodiment, control unit 22 comprises a pump (not shown) which dispenses the drug to the subject. Alternatively or additionally, control unit 22 is configured to facilitate the administration of the drug by controlling electrodes 24 to iontophoretically drive the drug into tissue of the subject.

In some embodiments, the outer surface of manual insertion device 20 is coated with a drug, and insertion of the device into the subject's vagina releases a portion of the drug. For some applications, the drug within reservoir 29 or on the surface of the manual insertion device includes a hormone, e.g., estrogen, or progesterone.

For some applications, manual insertion device 20 is configured to control a pH, and/or an odor of the vagina. Typically, a substance for controlling pH and/or odor is administered to the subject via reservoir 29. In some embodiments, the substance for controlling pH and/or odor comprises one or more of the following compositions: the compositions described in U.S. Pat. No. 6,479,045 by Bologna et al., which is incorporated herein by reference, clindamycin hydrochloride, which is marketed by Pfizer as Cleocin®, and/or metronidazole marketed as MetroGel® by Galderma. Alternatively, other compositions known in the art are administered via reservoir 29.

In some embodiments, water is delivered to the vagina via reservoir 29, and electrodes 24 are configured to electrolyze the water. The electrolysis of the water causes the release of oxygen inside the vagina, and the presence of oxygen acts to control odor, and/or control pH of the vagina. In some embodiments, a pharmaceutical substance or a odor-controlling and/or a pH-controlling substance is delivered to the vagina, via the reservoir, in addition to water. The electrodes are configured to electrolyze the water and release oxygen from the water. The electrolysis of the water further enhances the dispersal of the substance.

In an embodiment, the outer surface of the manual insertion device is coated with the substance for controlling pH and/or odor. For some applications, control unit 22 iontophoretically delivers the substance into the tissue of the vagina by driving an electrical current into the subject's vagina. In some embodiments, a direct current is driven into the vagina to facilitate iontophoretic delivery of the substance into the tissue, and (simultaneously or in alternation) a series of pulses is driven into the vagina to chronically increase nitric oxide production by tissue of the subject.

For some applications, the manual insertion device is configured to drive a current for treating urge incontinence. Typically, the current is driven into a pelvic muscle of the subject (e.g., a pelvic floor muscle), and has different signal parameters from those used to treat the female sexual dysfunction.

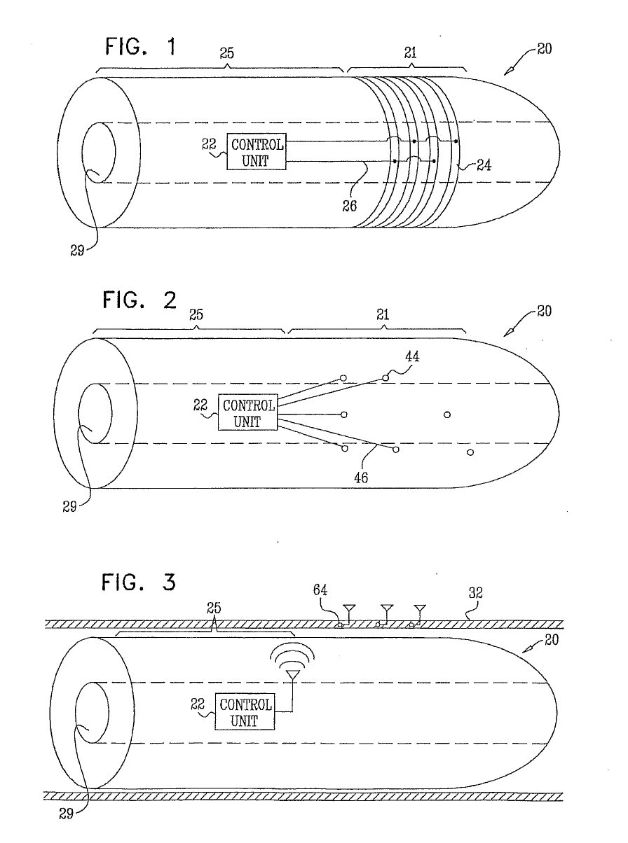

Reference is now made to FIG. 2, which is a schematic illustration of manual insertion device 20, in accordance with an alternative embodiment of the present invention. A plurality of electrodes 44 are disposed on the surface of the manual insertion device. One or more electrical leads 46 couples each of the respective electrodes to control unit 22. In all other aspects, manual insertion device 20 of FIG. 2 is generally similar to manual insertion device 20 of FIG. 1.

Reference is now made to FIG. 3, which is a schematic illustration of manual insertion device 20, in accordance with an alternative embodiment of the present invention. One or more electrode units 64, each typically comprising an electrode and an antenna or coil, are surgically implanted into tissue 32 of a pelvic site in or adjacent to the vagina. Control unit 22 is configured to wirelessly drive electrode units 64 to drive a current into the subject's vagina configured to chronically increase nitric oxide production by tissue of the subject. Alternatively or additionally, the control unit wirelessly drives the electrodes to drive a current into the pelvic site for a different purpose, for example, to facilitate administration of a drug, to control pH of the vagina, and/or to control an odor of the vagina. In all other aspects, manual insertion device 20 of FIG. 3 is generally similar to manual insertion device 20 of FIGS. 1 and 2.

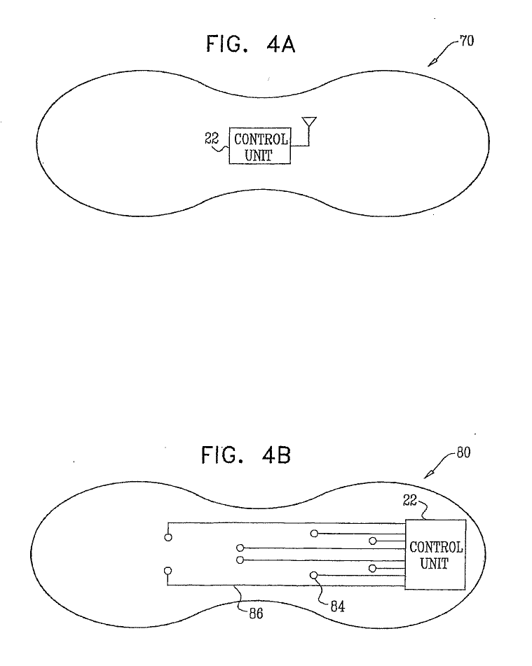

Reference is now made to FIG. 4A, which is a schematic illustration of a panty liner 70, in accordance with an alternative embodiment of the present invention. In a similar manner to that described with respect to FIG. 3, one or more electrode units 64, each typically comprising an electrode and an antenna or coil, are surgically implanted into tissue 32 of a pelvic site in or adjacent to the vagina. Control unit 22, disposed on the surface of panty liner 70, is configured to wirelessly drive electrode units 64 to drive a current into the subject's vagina, the current being configured to chronically increase nitric oxide production by tissue of the subject. In all other aspects, panty liner 70 of FIG. 4A is generally similar to manual insertion device 20 of FIGS. 1 and 2.

Reference is now made to FIG. 4B, which is a schematic illustration of a panty liner 80, in accordance with an alternative embodiment of the present invention. A plurality of electrodes 84 are disposed on the surface of the panty liner. One or more electrical leads 86 couple the electrodes to control unit 22. Control unit 22, disposed within or on the surface of panty liner 80, is configured to drive. electrodes 84 to drive a current into a vicinity of the subject's vagina. The panty liner is not inserted into the subject, but is placed outside the body of the subject. In all other respects, device 80 of FIG. 4B is generally similar to manual insertion device 20 of FIGS. 1 and 2 and/or panty liner 70 of FIG. 4A.

It is noted that although some embodiments of the present invention are described hereinabove with respect to a manual insertion device which facilitates the electrical stimulation described herein, in some embodiments of the present invention, techniques described hereinabove are applied to reduce nitric oxide production, rather than to increase nitric oxide production. For example, this may be done in order to reduce menorrhagia by reducing dilation of uterine blood vessels. For some applications, the current is a high frequency current, e.g., 30-100 Hz (such as 50-100 Hz), which inhibits nitric oxide synthase in cells of the cervix. In an embodiment, an implanted nerve cuff applies the current to reduce nitric oxide production to treat menorrhagia.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating female sexual dysfunction of a subject, comprising:
a manual insertion device configured to be inserted by the subject into a vagina of the subject and removed by the subject from the vagina of the subject;
at least one electrode coupled to the insertion device; and
a control unit, configured to chronically increase nitric oxide production by tissue of the subject by driving the electrode to drive an electric current into the vagina.

2. The apparatus according to claim 1, wherein a total length of the apparatus is 4-10 cm.

3. The apparatus according to claim 1, wherein the manual insertion device is configured to be inserted into the vagina of the subject in accordance with a schedule unrelated to timing of sexual intercourse of the subject.

4. The apparatus according to claim 3, wherein the manual insertion device is configured to be inserted into the vagina of the subject for between half an hour and two hours in a day, in accordance with a schedule unrelated to timing of sexual intercourse of the subject.

5. The apparatus according to claim 3, wherein the manual insertion device is configured to be inserted into the vagina of the subject for between two hours and five hours in a day, in accordance with a schedule unrelated to timing of sexual intercourse of the subject.

6. The apparatus according to claim 1, wherein the insertion device is configured to administer a drug.

7. (canceled)

8. The apparatus according to claim 1, wherein the insertion device is configured to control a pH of the vagina.

9-12. (canceled)

13. The apparatus according to claim 1, wherein the insertion device is configured to control an odor of the vagina.

14-17. (canceled)

18. The apparatus according to claim 1, wherein the control unit is configured to configure the electric current to have a frequency that is between 8 Hz and 20 Hz.

19. (canceled)

20. The apparatus according to claim 1, wherein the control unit is configured to configure the electric current to have an amplitude that is between 1 mA and 5 mA.

21. (canceled)

22. The apparatus according to claim 1, wherein the control unit is configured to treat urge incontinence by driving the electrode to drive an electric current into the vagina.

23-24. (canceled)

25. The apparatus according to claim 1, wherein the manual insertion device comprises a compressible material.

26. The apparatus according to claim 25, wherein the manual insertion device is configured:
to be inserted into the vagina by being compressed, and
upon having been inserted into the vagina, to conform to a shape of a vaginal cavity of the subject, by expanding.

27-29. (canceled)

30. A method for treating female sexual dysfunction of a subject, comprising:
driving an electric current from within a vagina of the subject; and
configuring the electric current to chronically increase nitric oxide production by tissue of the subject.

31. The method according to claim 30, further comprising inserting an electrode into the vagina in accordance with a schedule, wherein driving the electric current comprises driving the electric current via the electrode while the electrode is inserted.

32. The method according to claim 30, further comprising surgically implanting an electrode inside the vagina, and wherein driving the electric current comprises driving the electric current via the electrode while the electrode is implanted.

33. The method according to claim 30, wherein driving the electric current comprises inserting by the subject into the vagina an insertion device that is coupled to an electrode, and driving the electric current via the electrode.

34. The method according to claim 30, wherein driving the electric current comprises configuring the electric current to have a frequency that is between 8 Hz and 20 Hz.

35. (canceled)

36. The method according to claim 30, wherein driving the electric current comprises configuring the electric current to have an amplitude between 1 mA and 5 mA.

37. (canceled)

38. The method according to claim 30, further comprising controlling a pH of the vagina.

39. (canceled)

40. The method according to claim 38, wherein controlling the pH of the vagina comprises electrically controlling the pH.

41-43. (canceled)

44. The method according to claim 30, further comprising controlling an odor of the vagina.

45. The method according to claim 44, wherein controlling the odor of the vagina comprises electrically controlling the odor.

46-55. (canceled)

56. A method, comprising:
identifying a subject as suffering from female sexual dysfunction; and
treating the sexual dysfunction by driving an electric current from within a vagina of the subject in accordance with a schedule unrelated to timing of sexual intercourse of the subject.

57-69. (canceled)
